(19) United States
(12) Patent Application Publication
(10) Pub. No.: US 2012/0022320 A1
(43) Pub. Date: Jan. 26, 2012

(54) GASTRIC RING WITH A FITTING ROD

(75) Inventor: Leo Hendrickx, Morstel (BE)

(73) Assignee: Compagnie Europeenne d'Etude et de Recherche de Dispositifs pour l'implantation par Laparoscopie, Vienna (FR)

(21) Appl. No.: 13/146,109

(22) PCT Filed: Jan. 27, 2010
(86) PCT No.: PCT/EP2010/050946

§ 371 Date: Sep. 23, 2011

(30) Foreign Application Priority Data
Jan. 28, 2009 (FR) 0950530

(51) Int. Cl. A61F 2/00

(52) U.S. Cl. 600/37

(57) ABSTRACT

The invention relates to an implantable medical device (1) comprising a flexible band (2) intended for being placed around an organ to tightly bind the latter, said medical device being characterised in that it includes a positioning means (10) provided with a curved stiffening element with a rigidity greater than that of the flexible band, said positioning means (10) being designed to be inserted around the organ in order to substantially mark the path for placing said flexible band (2) around said organ. The invention can be used in the field of implantable medical devices.

The present invention pertains to the general field of implantable medical devices and more particularly to the field of implantable medical devices comprising a flexible band that is to be placed around an organ in order to tightly bind it.

The present invention pertains especially to surgical ring type devices and especially to gastric sphincters or rings designed to be implanted around the stomach or the esophagus in the context of a treatment for obesity.

There are known ways of treating patients affected by severe obesity where a gastric ring is implanted in patients to surround and tightly bind the stomach in order to limit the intake of food.

Such rings may be placed intact around the stomach in order to carry out a direct gastroplasty or after a “bypass” operation during which the stomach is surgically remodeled for the creation therein of a pouch of reduced size in order to prevent the subsequent expansion of said pouch.

To this end, there are known ways of introducing the gastric ring in open and relaxed form so that it can pass through the retro-gastric tissues.

This operation, which is generally performed under endoscopy, is nevertheless a delicate one inasmuch as a passage has to be opened by blind dissection of the retro-gastric tissues and then a grasping instrument has to be introduced through said passage so as to then grasp the end of the gastric ring with said grasping instrument and exert traction force on this ring in order to bring back the instrument through the passage, in hauling the ring in its train.

First of all, this sometimes requires that the practitioner should create a relatively large-sized passage which forces him to excessively devascularize the tissues, especially those of the stomach, and may in certain cases provoke post-operational complications.

Furthermore, although the engagement proper of the ring in the retro-gastric tissues generally takes only a few minutes, the grasping instrument must, on the contrary, be positioned well in advance and usually must remain in place during the major part of the operation.

Now, since this instrument has to be controlled by a proximal portion situated outside the patient, a section of said instrument passes constantly through the abdominal wall, via a catheter. This transcutaneous communication is a point of leakage through which escapes the gas used to inflate the abdominal cavity during the operation. The operation then requires a fairly high flow rate and/or supply pressure and leads to excess consumption of inflating gas.

The objects assigned to the present invention are therefore aimed at overcoming the drawbacks enumerated here above and proposing a novel implantable medical device designed to be placed around an organ to tightly bind this organ, this device being particularly simple and reliable in its implantation, especially by laparoscopy.

Another object assigned to the invention is aimed at proposing a novel implantable medical device that facilitates the surgical gestures needed for its implantation.

Another object assigned to the present invention is aimed at proposing an implantable medical device that substantially reduces the risks of per-operational accidents, as well as the trauma suffered by the patient.

Another object assigned to the invention is aimed at proposing a novel implantable medical device that is versatile and capable of being adapted on a case-by-case basis to the anatomical configuration of the patient under operation.

Another object assigned to the present invention proposes a novel implantable medical device having high ergonomy that makes it easier to handle by the practitioner.

Another object assigned to the invention is aimed at proposing a novel implantable medical device that is relatively simple and compact in its design.

Another object assigned to the invention is aimed at proposing a novel implantable medical device having high stability once implanted.

Finally, another object assigned to the invention is aimed at proposing a novel implantable medical device adapted to the treatment of obesity.

The objects assigned to the invention are achieved by means of an implantable medical device having a flexible band that is to be placed around an organ in order to tightly bind this organ, said medical device being characterized in that it comprises a positioning means provided with a curved stiffening element having a rigidity greater than that of the flexible band, said positioning means being designed to be engaged around the organ in order to substantially mark out the path along which said flexible band is placed around said organ.

Other objects, features and advantages of the invention shall appear in greater detail from the following description as well as by means of the appended drawings which are given purely by way of illustration and on a non-exhaustive basis. Of these drawings:

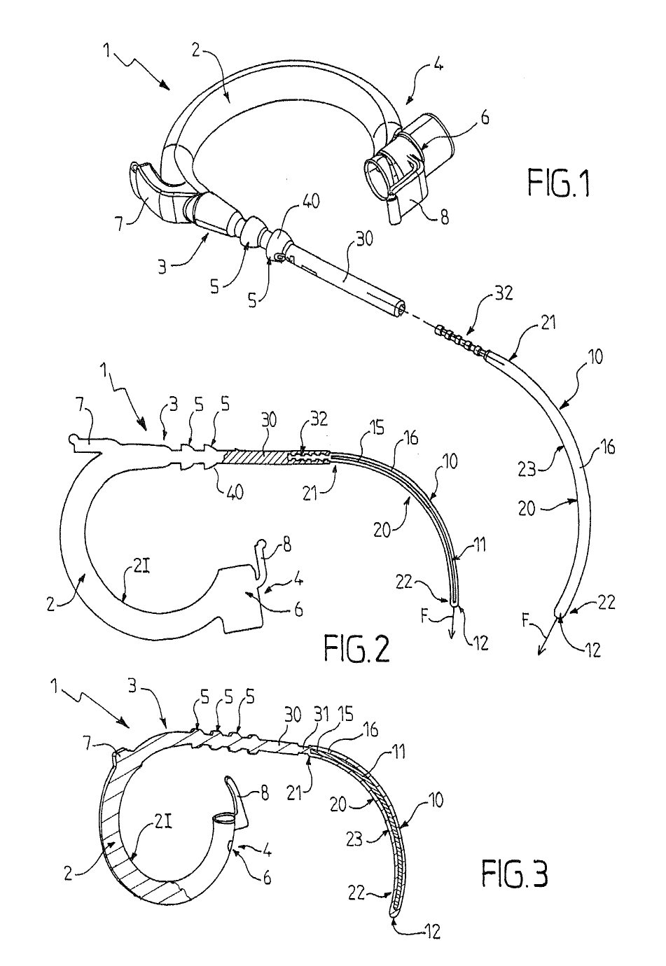

FIG. 1 provides an illustration, in an exploded view in perspective, of an alternative embodiment of the implantable medical device according to the invention.

FIG. 2 is an illustration, in a lateral view of a partial sagittal section, of the medical device shown in FIG. 1.

FIG. 3 is an illustration, in a lateral view of a partial sagittal section, of a second alternative embodiment of the implantable medical device according to the invention.

The present invention generally concerns an implantable medical device 1 comprising a flexible band 2 that is to be placed around an organ (not shown) in order to tightly bind or grip this organ.

Preferably, the device 1 according to the invention is designed to be placed around an organ forming a pouch or a duct so as to limit or reduce the section of passage of this organ.

According to one alternative embodiment, said device 1 may be laid out to form a sphincter for regulating the blood flow and/or for treating urinary or fecal incontinence.

However, preferably, said device 1 is a gastric ring intended for the treatment of obesity, designed to be positioned on the esophagus or on the stomach and especially designed to constitute a gastric ring specifically adapted to being placed on the stomach pouch resulting from a surgical bypass operation in order to counter the post-operational expansion of said stomach pouch.

Besides, although it is possible, without departing from the framework of the invention, to envisage a case where the flexible band 2 forms a sort of saddle designed to partially surround the concerned organ, said flexible band 2 will preferably be long enough to contain substantially the entire perimeter of said organ and especially to be closed in on itself substantially at its ends 3, 4 so as to be capable of forming a closed loop surrounding the organ.

To this end, the flexible band is preferably provided with locking means 5, 6 designed to hold it in a closed configuration, said locking means comprising for example male elements 5 such as pins, intended for cooperating, for example by being clipped on, with a corresponding female element 6 such as a sleeve.

Advantageously, the locking means are reversible to enable the alternate closure and opening of the ring formed by the flexible band.

It can furthermore be noted that the locking means 5, 6 can advantageously be designed to enable an adjustment of the length of the internal perimeter of the flexible band and may especially comprise a succession of male elements 5 aligned in a row so as to form several possible closing notches as illustrated in FIGS. 1 to 3.

According to one alternative embodiment not shown, the internal face 21 of the flexible band 2, which is to come into contact with the organ, may comprise one or more inflatable pouches, designed to be filled with a filling fluid, such as air or physiological serum, in order to provide for an adjustable constriction of the organ. The present invention also pertains to an inflatable ring, in particular a gastroplasty ring.

In particular, the device 1 may comprise an annular binding pouch that extends substantially throughout the length of the flexible band so as to ensure a substantially radial centripetal compression of said organ.

The device 1 may also comprise one or more grasping tabs 7, 8 aimed at making it easier to handle said device, especially when it is implanted and/or for enabling the actuation of the locking means.

According to one major characteristic of the invention, the device 1 comprises a positioning means 10 provided with a curved stiffening element 11 which has a rigidity greater than that of the flexible band 2, said positioning means being designed to be engaged around the organ in order to substantially mark out the trajectory by which said flexible band 2 is placed around said organ.

Thus, as understood in the invention, the device 1 has an incorporated positioning means 10 carried by the device 1 and forming an integral part of said device during the operation and is designed to be entirely introduced beneath the skin, for example into the abdominal cavity, fixedly with the flexible band in such a way that the device and more particularly the totality of its positioning means can be handled freely inside the body in the vicinity of the organ.

Furthermore, although it can be envisaged that the junction of the positioning means 10 with the rest of the device permits the captive play, of an amplitude limited by construction, of said positioning means 10 relatively to the rest of the device 1, the positioning means is preferably fixed to the device, for example through the flexible band, so as to form a fixedly joined assembly with this means, advantageously implantable and capable of being handled as a single piece beneath the skin.

Advantageously, the positioning means 10 of the invention is designed to be introduced into the tissues, especially the retro-gastric tissues, so as to go around the organ until it reappears on the visible face of this organ, enabling the practitioner to determine and pre-visualize the position that will be occupied by the flexible band around the organ before truly engaging this band around the organ.

To this end, the curvature of the stiffening element 11 and therefore that of the positioning means 10 is advantageously chosen so as to enable said positioning means to initiate a sharp turn around the organ until it is introduced into the tissues, thus making it possible to easily circumvent said organ without any risk of perforating the underlying tissues.

Naturally, the stiffening element 11 advantageously has rigidity sufficient to enable the positioning means 10 to be inserted and not get deformed in an untimely way when said positioning means is drawn into the tissues and maneuvered therein by the practitioner.

Thus, the device 1 of the invention facilitates the introduction and placing of the flexible band 2, which in itself is too slack to be capable of being introduced frontally into the tissues, while allowing the device, during and after the positioning, to preserve the flexibility needed for it to be closed around the organ and function normally.

Preferably, the positioning means 10 comprise a penetration means 12 designed to enable the creation of a passage in the vicinity of the organ in perforating the tissues.

In other words, the positioning means 10 and more generally the device 1 is advantageously adapted to being capable by itself, when put into place, of causing incisions and/or tearing biological tissues and especially the healthy biological tissues that surround the organ.

The practitioner can then directly use the positioning means 10 to drill a tunnel by driving said positioning means in a frontward motion F under stress which pushes it into this tunnel while gradually separating the mass of tissue.

Thus, the positioning means 10 according to the invention can advantageously open a passage by frontal penetration into the tissues and do not necessarily require the prior making of a suitable passage with a distinct surgical instrument.

Consequently, such a self-drilling positioning means 10 gives the device 1 a certain degree of autonomy during the positioning and simplifies the surgeon's gestures by eliminating the step of dissection prior to this operation phase.

Naturally, the positioning means 10 and more particularly the stiffening element 11 is rigid enough, i.e. sufficiently resistant to bending forces to substantially maintain its original shape and especially the predetermined curvature of the positioning means 10 when these means are force-inserted into the tissues to pierce the passage.

Preferably, the penetration means 12 are formed by a smoothened or rounded tip as illustrated in FIGS. 1 to 3.

Advantageously, an arrangement of this kind reduces per-operational risks such as accidental perforation of the wall of the organ and limits the extent of trauma undergone by the tissues during the creation of the passage.

Preferably, although the stiffening element is rigid enough to withstand especially buckling forces when it is being introduced, said stiffening element 11 is preferably plastically configurable so as to enable the adjustment of the geometry of the positioning means 10 before these means are engaged around the organ.

In other words, it is advantageously possible for the practitioner to modify the spatial layout of the stiffening element 11 and more generally that of the positioning means 10 before its introduction around the organ so as to give it the desired shape and curvature given the particular anatomy of the patient being treated.

In particular, the stiffening element 11 could advantageously adopt and maintain a curvature that is pronounced to a greater or lesser extent and is modifiable at leisure by the practitioner depending on whether he or she plastically incurvates or straightens out the positioning means 10.

Naturally, those skilled in the art will be capable of determining the constituent materials of the stiffening element 11 as well as the geometry and the dimensions of this element, making it possible to meet the twofold requirement of resistance to buckling forces during penetration into the tissues and of plastic flexibility enabling the positioning means to be modeled on site and especially in the abdominal cavity before it is inserted behind the organ.

Advantageously, the stiffening element 11 could be formed by means of shape memory materials or super-elastic materials in such a way that said stiffening element could alternately adopt a straightened configuration, a straightened and substantially rectilinear configuration, enabling its passage into a transcutaneous insertion trocar device, and a curved functional configuration. The changing from one configuration to another can advantageously be done spontaneously as the case may be, either under the effect of a change in temperature and especially a heating of body temperature (for a shape memory material) or under the effect of the relaxation of a mechanical straightening force exerted during introduction beneath the skin by the trocar or any unspecified accessory (for a super-elastic material).

According to a preferred characteristic illustrated in FIGS. 2 and 3, which may constitute a full-fledged invention on its own, the positioning means 10 comprises a rigid or semi-rigid core 15 forming the stiffening element 11, said core 15 being housed in a sheath 16 made of flexible biocompatible material.

Thus advantageously, the positioning means 10 may take the form of a coated needle, i.e. a composite structure in which the stiffening element or elements are clad with a flexible and advantageously biocompatible coating.

By way of an example, the core 15 could be made out of ABS or polypropylene type plastic material or any other non-brittle plastic material possessing appropriate rigidity and resistance to strain-hardening.

According to another alternative embodiment, the core 15 could be made out of titanium, stainless steel, or any other ductile metal that is sufficiently easy to shape in a plastic manner, but at the same time has high stiffness and therefore a low tendency to elastic deformation.

The sheath 16 for its part could be made out of a silicon type elastomer, EPDM, polyurethane or any other flexible and biocompatible material.

Besides, the positioning means 10 is preferably laid out so that that it can be disengaged from the vicinity of the organ in a forward motion F having the same sense as the motion by which it is engaged around said organ in order to position the flexible band 2.

In other words, the extraction of the positioning means 10 from the operational area when said positioning means is still beneath the skin, can advantageously be done in continuing the movement initiated when this means was introduced into the tissues and when the organ was circumvented, and this can be done without its being necessary to make the positioning means 10 turn back when it is engaged in the tissues.

Thus, the invention prevents said tissues from being subjected to alternating stresses exposing them to risks of wrenching and tearing.

Furthermore, such a characteristic gives the device 1 high ergonomy, since its positioning can be done in an overall sliding motion which corresponds to a fairly “gentle” and natural gesture for the practitioner.

Furthermore, the positioning means 10 is preferably connected to one of the ends 3, 4 of the flexible band 2 and extends so as to protrude out of said flexible band 2 substantially in the longitudinal prolongation of this band 2.

Within the meaning of the invention, the longitudinal direction of the band corresponds to the direction that joins the first end 3 to the second end 4 of said flexible band and substantially follows the incurvated contour adopted by the flexible band when it substantially matches the external wall of the organ that it tightly binds.

More particularly, the positioning means 10 is advantageously laid out so as to extend from the first end 3 of the flexible band 2, beyond the internal face 21 of this band and opposite the second end 4 of said flexible band 2.

Thus, the positioning means 10 and more particularly the stiffening element 11 are advantageously yoked to the band, upstream from it and in a non-overlapping manner, so as to enable the successive engagement of the positioning means 10 and then of the flexible band 2 in the tissues.

Preferably, the positioning means 10 is integrally situated on only one side of the flexible band and in a particularly preferred way at the end of this band, in a substantially centered manner relatively to its cross-section.

More particularly, as illustrated in FIGS. 1 to 3, the positioning means 10 can be formed by a rod 20 that extends between a foot 21 connected to the flexible band 2 and a free head 22 which carries the penetration means 12.

Preferably, the foot 21 is connected to the head 22 by means of an intermediate section 23 which contains or is formed by the stiffening element 11 in such a way that a penetration force exerted by the foot 21 can be transmitted by said intermediate section to the head 22 which rests against the tissues.

Thus, the positioning means 10 is advantageously designed so as to be grasped, handled and guided by its foot 21 which forms a sort of sleeve when the head 22, which remains free, is pushed into the tissues.

Advantageously, the layout of the positioning means 4 according to the invention enables a practitioner in a first stage to push the rod 20 substantially from the head 22 to the foot 21 into the tissues which surround the organ, until said head 22 is made to emerge on the organ opposite the entry side of said rod, and then, in a second stage, to exert a traction force on the emerging part of the head 22 in such a way that the rod 20 is gradually extracted from the passage that it has formed so as to be gradually replaced by the flexible band 2, this band thus penetrating the passage following the positioning means 10 that precedes it.

Advantageously, the rigidity of the intermediate section 23 and of the foot 21 enables a grasping and handling of the positioning means 10 itself but also preferably of the flexible band 2 to which it is attached

According to one preferred embodiment illustrated in FIGS. 1 to 3, the rod 20 is cylindrical and has a circular section, which is preferably substantially constant, giving it low traumatizing capacity with respect to the tissues when it moves in contact with these tissues.

Furthermore, such an arrangement gives the device 1 and its positioning means 10 a particularly simple and compact structure that costs little to manufacture.

According to one embodiment illustrated in FIG. 3, the positioning means 10 forms one piece with the flexible band 2.

More particularly, the foot 21 of the rod 20 and more particularly the sheath 16 could be shaped as one piece with a tip 30, which is advantageously cylindrical, that prolongs the flexible band 2 and forms the end of the male locking means 5.

More particularly, the positioning means 10 has then a preferred separation zone 31 at its junction with the flexible band 2, such as a scored zone, designed to facilitate the separation of the positioning means 10 and the flexible band 2 after this band has been positioned.

As shown in FIG. 3, the preferred separation zone 31 may consist of a thinned or compressed portion forming a preferred breaking or cut-off point enabling the practitioner to remove the rod 20 by ablation after it has been used to introduce the flexible band 2 around the organ.

Thus, after the device 1 has been placed and locked around the organ, it has almost no protruding parts left and the residual space that it occupies is particularly limited, thus restricting the risks of post-operational complications and improving the patient's comfort.

However, according to one preferred embodiment of the invention, the positioning means 10 is provided with reversible fastening means 32 designed to enable it to be alternately connected to the device 1 and separated from the device 1 under the practitioner's control.

In other words, the fastening means 32 advantageously give the positioning means 10 and more particularly the rod 20 a detachable character and give the device 1 a modular character.

Said reversible fastening means 32 could, by way of an example, include a toothed protuberance extending the foot 21 of the rod opposite the head 22, and intended for working together, by a clip-on means, with a notched housing made for this purpose at the end of the tip 30 to enable the assembling of the device 1 as illustrated in FIGS. 1 and 2.

Advantageously, the rod 20 and more comprehensively the positioning means 10 could thus form an outlet for a projecting element that is adaptable at the end of the flexible band 1 and capable of being fixed, preferably in a substantially centered manner, in the prolongation of said flexible band 2.

In this respect, it can be noted that the invention also pertains to the positioning means 10, detachable per se, and especially the rod 20 provided by the core 15 and its sheath 16 and its reversible fastening means 32.

Advantageously, it can be envisaged that the fastening means of such a positioning means 10 will be designed in such a way that the positioning means 10 can either be joined directly to the end of the flexible band 2, i.e. in the tip 30 or, if the device is provided with an inflation pouch connected to a filling catheter designed to place said pouch in communication with a fluid container of the implantable site type, joined to the free end of said catheter.

Furthermore, according to another alternative embodiment, if the device 1 is provided with a filling catheter connected to or intended for being connected to the flexible band 2 substantially at one of the ends of this band 2, the positioning means 10 and more particularly the stiffening element 11 could protrude, and preferably originate at the other, advantageously free, end of the device 1, substantially opposite said catheter.

According to one alternative embodiment, the positioning means 10 and more particularly the stiffening element 11 can advantageously be mounted on or originate in proximity to that one of the ends 3, 4 of the flexible band 2 with the smallest space requirement, i.e. that has the transverse size or cross-section with the smallest span, and more particularly can advantageously be fixed to the first end 3 which bears the male element 5 of the locking means upstream to these locking means in considering the planned sense of the forward motion F.

Advantageously, an arrangement of this kind makes it possible to draw along the flexible band 2 in the retro-gastric passage in a sense of introduction that is relatively non-traumatic i.e. in making to make it only the first end 3, which is the most compact end and therefore the least traumatizing end, “travel” through said retro-gastric passage during the positioning of the device 1, while the opposite second end 4, which is bulkier, remains withdrawn without getting engaged in said passage. Thus, the tissues of said passage are constrained only to the precise extent necessary and sufficient for the positioning of the device.

Furthermore, the positioning means 10 preferably have a cross-section smaller than that of the flexible band 2.

More particularly, the rod 20 will preferably have a diameter smaller than the smallest overall transverse size of the flexible band 2, as measured perpendicularly to the longitudinal direction of this band.

Thus, the positioning means 10 do not have any excess thickness relatively to the band in such a way that it does not unnecessarily hollow out a tunnel that is oversized relatively to the capacity needed to receive the flexible band.

This advantageously prevents trauma and especially devascularization from occurring in an excessively large volume of tissue and thus gives the device relatively low invasiveness.

Furthermore, the fact that the practitioner can use the positioning means 10 of the invention to hollow out a passage with a narrower section of the cross-section of the flexible band 2 advantageously enables the force-insertion of said flexible band 2 into said passage in constricting this band by a natural elastic return of the tissues, which improves the holding of said flexible band 2 and gives the implanted device high stability.

Naturally, the transition zone between the positioning means 10 and the flexible band 2 could advantageously comprise spreader means 40 making it easier to spread apart the tissues that demarcate the passage during the insertion of the flexible band 2.

In this respect, said spreader means 40 could advantageously be constituted by the inclined faces of the pins 5 starting from the tip 30 and forming the male locking means.

Preferably, the positioning means has a length substantially equal to that of the stiffening element 11.

The length of the positioning means 10 and/or that of the stiffening element 11 is furthermore preferably included between 50% and 100% of the length of the flexible band and in a particularly preferable way included between 60% and 70% of the length of said flexible band, the length of the flexible band being herein considered to be the length of its contact zone, i.e. substantially the value of the perimeter of the organ. Advantageously, the length of the positioning means is greater than the external semi-perimeter of the organ around which the flexible band has to be implanted.

Furthermore, the positioning means 10, when in the functional configuration, is preferably curved substantially throughout its length, preferably continuously in a curvature of the same sign.

Preferably, the positioning means is entirely situated so as to be projecting out of the flexible band 2 and forms an extension of invariant length relatively to this flexible band 2.

Furthermore, the external diameter of the sheath 16 preferably ranges from 2 mm to 5 mm while the diameter of the stiffening element 1, which preferably takes the form of a rod or a thick rope is for its part substantially between 1.5 mm and 3 mm.

Naturally, the present invention also pertains to a surgical method for the implanting of a medical device which shall now be described in greater detail with reference to the preferred alternative embodiment of the device 1 illustrated in FIGS. 1 and 2.

First of all, said surgical method preferably includes a mounting step (a) during which the practitioner integrates the positioning means 10 into the device 1 in attaching and fixing the rod 20, preferably by fitting in the fastening means 32 to one of the ends 3 of the flexible band 2 or, as the case may be, to the free end of the catheter connected to said flexible band.

Preferably, said method then comprises an adjustment step (b) during which the practitioner elastically deforms the rod 20 and more particularly the stiffening element 11 in order to model the positioning means 10 and give it a geometry and especially a curvature suited to the dimensions and shape of the organ to be treated.

To this end, the practitioner may in particular use the plasticity of the stiffening means 11 by exerting a bending force on the intermediate section 23 in order to curve it or on the contrary in order to straighten it.

Advantageously, the adjusting step (b) can be done either directly by hand or indirectly, using laparoscopy instruments, when the device 1 is entirely beneath the skin, especially in the abdominal cavity.

Once the desired shape has been obtained, the method continues with an engagement step (c) during which the practitioner makes the positioning means 10 penetrate into the tissues that are contiguous to the organ and especially into the tissues situated behind said organ, and causes the positioning means 10 to move forward through said tissues so as to make it circumvent the organ until the head 22 emerges from said tissues substantially opposite the side by which the positioning means 10 is introduced relatively to the organ.

To this end, the practitioner can advantageously grasp the rod 20 by its foot 21 and exert a thrust force on said rod that is transmitted to the head 22 by the intermediate section 23 so that the head 22 compresses the tissues and forces a passage through these tissues in a forward movement F.

Advantageously, the engagement step (c) is simultaneously accompanied by a perforation sub-step (c1) during which the practitioner pierces and separates the tissues situated before the head 22 by means of the penetration means 12 in the sense of the forward motion F as and when the rod 20 moves forward in order to clear a passage in the mass of tissue.

In other words, the practitioner can advantageously introduce the positioning means 10 directly by force into the tissues in order to draw said positioning means around the organ and if necessary guide it along feeling his way around.

Advantageously, the rigidity of the stiffening element enables it to lastingly maintain the conformation of the positioning means 10 chosen by the practitioner, especially to counter the buckling forces resulting from the resistance to penetration put up against it by the tissues.

Once the free end of the positioning means 10, i.e. the head 22, has emerged from the tissues, i.e. once the organ has been “hooked”, the practitioner can visually ascertain that the position of the positioning means relatively to the organ substantially corresponds to the configuration in which he wishes to place the flexible band around said organ.

Advantageously, the length of the positioning means 10 is sufficient for this means to substantially mark out the entire path for placing the flexible band around the concealed, portion i.e. the non-apparent portion, of the perimeter of the organ.

If necessary, the practitioner can maneuver the positioning means or even make it take the return path at least partly in order to then reintroduce it around the organ and bring it, by iteration, to a position that he considers to be appropriate.

The practitioner then performs a step (d) of extraction during which he grasps the positioning means 10, for example by gripping the emerging head 22 by means of a clamp, and then exerts a traction force on said positioning means 10 so as to gradually withdraw it from the tissues, in a motion that is in the same sense as the forward motion F which had enabled the insertion of the positioning means 10.

Advantageously, the extraction of the positioning means 10 prompts a mechanical pull on the rest of the device 1 by traction on the end of the flexible band 2, either directly at the tip 30 or indirectly at the catheter which acts like a traction cable.

Under the effect of this traction, the flexible band 2 appears at the entry to the passage hollowed out by the positioning means into which it penetrates, the spreader means 40 deforming said passage to widen it and facilitate the progress in succession of the rest of the flexible band 2.

Said flexible band 2 is thus drawn by the positioning means 10 and guided by the passage in the wake of said positioning means until it has substantially gone around the organ and until its first end 3 emerges from the passage.

The extraction step (d) is thus accompanied by a sub-step in which the flexible band 2 is gradually substituted for the positioning means 10 in the passage preformed by this positioning means 10.

Advantageously, the second end 4 of the flexible band 2 is not engaged in the passage and remains constantly emergent on the side of the organ where said band had been introduced.

The practitioner can then proceed to a separation step (e) during which he disconnects and moves the positioning means 10 away from the device 1 and more particularly from the flexible band 2 in unlocking the fastening means 32 or, as the case may be, in sectioning the junction between the positioning means 10 and the flexible band 2 at the preferred separation zone 31.

Advantageously, the flexible band 2 is held by itself in place around the organ during this separation step, through the elastic stress exerted on it by the tissues which constitute the wall of the passage, said flexible band 2 being thus sheathed by a natural and vascularized sleeve of tissue.

Besides, before the separation step (e) the practitioner can use the positioning means 10 and more particularly thread them into the sleeve forming the is female locking means 6 in order to engage the male locking means 5 into this female locking means and thus form the device 1 by locking it into position around the organ.

When the positioning means is fixed to the catheter, it can then be disconnected from the catheter and replaced by the implantable site.

Naturally, this step for closing the ring could also be achieved, as the case may be, after the separation step (e).

Thus, the implantable medical device of the invention advantageously forms an autonomous device which can be positioned simply and reliably by its own means requiring a minimum number of surgical gestures, thus reducing the difficulty, time and risks of the operation.

Furthermore, the device 1 according to the invention advantageously has a simple, compact and substantially non-traumatic structure.

1. Implantable medical device comprising
a flexible band configured to be placed around an organ in order to tightly bind the organ, and
a positioning device provided with a curved stiffening element having a rigidity greater than that of the flexible band, said positioning device being designed to be engaged around the organ in order to substantially mark out the path along which said flexible band is placed around said organ.

2. Medical device according to claim 1, wherein the positioning device comprises a penetration member designed to enable the creation of a passage in the vicinity of the organ in perforating the tissues.

3. Medical device according to claim 2, wherein the penetration member is formed by a smoothened or rounded tip.

4. Medical device according to claim 1, wherein the stiffening element is plastically configurable so as to enable the adjustment of the geometry of the positioning device before this means is engaged around the organ.

5. Medical device according to claim 1, wherein the positioning device is preferably laid out so that that it can be disengaged from the vicinity of the organ in a forward motion F having the same sense as the motion by which it is engaged around said organ in order to position the flexible band.

6. Medical device according to claim 1, wherein the positioning device is connected to one of the ends of the flexible band and extends so as to protrude out of said flexible band substantially in the longitudinal prolongation of this band.

7. Medical device according to claim 1, wherein the stiffening element has a length between 50% and 100% of the length of the flexible band.

8. Medical device according to claim 1, wherein the positioning device has a cross-section smaller than that of the flexible band.

9. Medical device according to claim 1, wherein the positioning device is formed by a cylindrical rod of substantially constant section.

10. Medical device according to claim 1, wherein the positioning device comprises a rigid or semi-rigid core forming a stiffening element, said core being housed in a sheath made of flexible biocompatible material.

11. Medical device according to claim 1, wherein the positioning device forms one piece with the flexible band.

12. Medical device according to claim 11, wherein the positioning device, at the level of its junction with the flexible band, has a preferred separation zone, designed to facilitate its separation from the flexible band after this band has been positioned.

13. Medical device according to claim 1, wherein the positioning device is provided with a reversible fastener designed to enable it to be alternately connected to the device and separated from said device.

14. Medical device according to claim 1, wherein it constitutes a gastric ring for the treatment of obesity.

15. Medical device according to claim 1, wherein the stiffening element has a length between 60% and 70% of the length of said flexible band.

16. Medical device according to claim 11, wherein the preferred separation zone includes a scored zone.
